(19) United States
(12) Patent Application Publication
(10) Pub. No.: US 2026/0140071 A1
(43) Pub. Date: May 21, 2026

(54) PHASE-CONTRAST ENHANCED X-RAY IMAGING SYSTEMS

(71) Applicant: Teledyne DALSA B.V., Eindhoven (NL)

(72) Inventors: Jonathan E. Snyder, Park City, UT (US); Andriy Lomako, Waterloo (CA); James J. Miller, Tavistock (CA)

(21) Appl. No.: 18/950,604

(22) Filed: Nov. 18, 2024

(51) Int. Cl. G01N 23/041; G01N 23/083; G01N 23/18; G01N 33/483

(52) U.S. Cl. CPC G01N 23/041; G01N 23/083; G01N 23/18; G01N 33/4833; G01N 2800/7028

(57) ABSTRACT

A system for generating a phase-contrast enhanced X-ray image is provided. The system comprises a substrate, an X-ray source, an X-ray detector, and a control circuit. The substrate is capable to hold a specimen. The X-ray source is positioned a first distance from the substrate and capable to emit X-ray electromagnetic radiation towards the substrate. The X-ray detector is positioned a second distance from the substrate and opposing the X-ray source. The second distance enables X-ray electromagnetic radiation emitted by the X-ray source to form an interference pattern. The X-ray detector is capable to receive X-ray electromagnetic radiation including the interference pattern. The control circuit is in signal communication with the X-ray source and the X-ray detector. The control circuit is capable to generate a phase-contrast enhanced X-ray image of the specimen based on the received X-ray electromagnetic radiation and the interference pattern.

### FIELD

This disclosure relates generally to systems for generating phase-contrast enhanced X-ray images.

### BACKGROUND

Diagnosing breast cancer involves various examination techniques directed to supporting early and accurate cancer detection, including, for example, X-ray imaging. There are challenges with identifying breast cancer early and efficiently.

### SUMMARY

In one general aspect, the present disclosure is directed to a phase-contrast enhanced X-ray imaging system. The system comprises a substrate, an X-ray source, an X-ray detector, and a control circuit. The substrate is capable to hold a specimen and comprises a first side and a second side. The X-ray source is positioned a first distance from a first side of the substrate and capable to emit X-ray electromagnetic radiation towards the first side of the substrate. The X-ray detector is positioned a second distance from a first side of the substrate and opposing the X-ray source. The second distance enables X-ray electromagnetic radiation emitted by the X-ray source to form an interference pattern. The X-ray detector is capable to receive X-ray electromagnetic radiation including the interference pattern. The control circuit is in signal communication with the X-ray source and the X-ray detector. The control circuit is capable to generate a phase-contrast enhanced X-ray image of the specimen based on the received X-ray electromagnetic radiation from the X-ray detector and the interference pattern.

Another general aspect of the present disclosure is directed to a phase-contrast enhanced X-ray imaging system. The system comprises a substrate, an X-ray source, an X-ray detector, and a control circuit. The substrate is capable to hold a specimen and comprises a first side and a second side. The X-ray source comprises a focal spot size. The X-ray source is positioned a first distance from a first side of the substrate and capable to emit X-ray electromagnetic radiation towards the first side of the substrate. The X-ray detector comprises a spatial resolution. The X-ray detector is positioned a second distance from a first side of the substrate and opposing the X-ray source. The second distance enables X-ray electromagnetic radiation emitted by the X-ray source to form an interference pattern. The X-ray detector is capable to receive the X-ray electromagnetic radiation including the interference pattern. The control circuit is in signal communication with the X-ray source and the X-ray detector. The focal spot size, the second distance, and the spatial resolution of the X-ray detector are capable to enable the control circuit to generate a phase-contrast enhanced X-ray image of the specimen.

Although the present disclosure relates to different aspects and embodiments, it is understood that the different aspects and embodiments disclosed herein can be integrated, combined, or used together as a combination system, or in part, as separate components, devices, and systems, as appropriate. Thus, each embodiment disclosed herein can be incorporated in each of the aspects to varying degrees as appropriate for a given implementation.

These and other features of the applicant's teachings are set forth herein.

### BRIEF DESCRIPTION OF THE FIGURES

Unless specified otherwise, the accompanying drawings illustrate aspects of the innovations described herein. Referring to the drawings, wherein like numerals refer to like parts throughout the several views and this specification, several embodiments of presently disclosed principles are illustrated by way of example, and not by way of limitation. The drawings are not intended to be to scale. A more complete understanding of the disclosure may be realized by reference to the accompanying drawings in which:

FIG. 1 is a schematic diagram of a non-limiting embodiment of a system for generating a phase-contrast enhanced X-ray image in accordance with the present disclosure;

FIG. 2 is an X-ray image of a subject containing embedded objects representative of microcalcifications within biopsied breast tissue, produced by prior imaging systems; and

FIG. 3 is a non-limiting embodiment of a phase-contrast enhanced X-ray image of a subject containing embedded objects representative of microcalcifications within biopsied breast tissue, in accordance with the present disclosure.

### DETAILED DESCRIPTION

After an initial breast cancer screening, a biopsy procedure where a sample of breast tissue is removed from the breast and analyzed in-vitro by a physician may be performed. Various systems for analyzing biopsied tissue are capable to obtain an X-ray image of the sample.

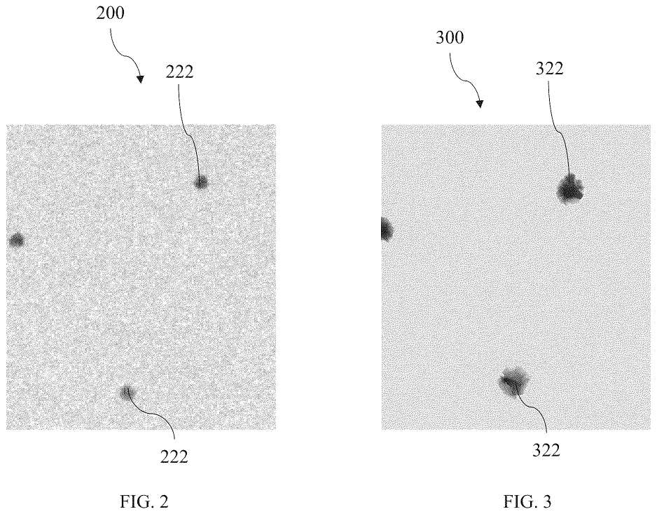

A physician can use the images to look for microcalcifications within the sample. Microcalcifications are small calcium-rich formations in breast tissue. An example of an X-ray image 200 comprising microcalcifications 222 representative of those found in breast tissue produced from prior imaging systems is shown in FIG. 2. Depending on the size, shape, grouping, composition, and other features in connection with the subject's personal demographics, microcalcifications may be early indicators of cancer development. However, not all microcalcifications are necessarily indicative of potential cancer development.

Microcalcifications can be on the order of 100 μm in diameter. While microcalcifications may be generally visible in prior images, the quality of the features resolved in the images may not be sufficient to classify the microcalcifications. Because of microcalcifications' small size, the present inventors determined that enhancing the spatial resolution of the X-ray imaging chain employed in breast tissue imaging systems can enhance the ability to classify the microcalcifications, which can enhance the ability of a physician to determine the relevance of the microcalcifications.

Traditional phase-contrast imaging techniques are expensive, as they require substantial investment into dedicated high performance X-ray sources, X-ray detectors, and other accessories. As such, traditional phase-contrast imaging is not currently suitable for routine clinical use and mass deployment in breast cancer diagnostic programs.

Thus, the present inventors provide a phase-contrast enhanced X-ray imaging system. The system comprises a substrate, an X-ray source, an X-ray detector, and a control circuit. The substrate is capable to hold a specimen and comprises a first side and a second side. The X-ray source is positioned a first distance from the first side of the substrate and capable to emit X-ray electromagnetic radiation towards the first side of the substrate. The X-ray detector is positioned a second distance from a first side of the substrate and opposing the X-ray source. The second distance enables X-ray electromagnetic radiation emitted by the X-ray source and refracted by the specimen to form an interference pattern. The X-ray detector is capable to receive X-ray electromagnetic radiation including the interference pattern. The control circuit is in signal communication with the X-ray source and the X-ray detector. The control circuit is capable to generate a phase-contrast enhanced X-ray image of the specimen based on the received X-ray electromagnetic radiation passing through the specimen and received by the X-ray detector.

The phase-contrast enhanced X-ray imaging system according to the present disclosure utilizes phase-contrast interference patterns to enhance X-ray images. For example, the present disclosure may combine both absorption and interference patterns in the same image. The interference pattern can enhance the typical X-ray absorption images enabling the resolution of additional features in the images, which can be used to enhance classification of the microcalcifications. The present inventors determined that the relationship between the focal spot size of the X-ray source, the spatial resolution of the X-ray detector, and the distance between the X-ray detector and the specimen being imaged can be balanced to enable an X-ray imaging system to produce a phase-contrast enhanced X-ray image of the specimen.

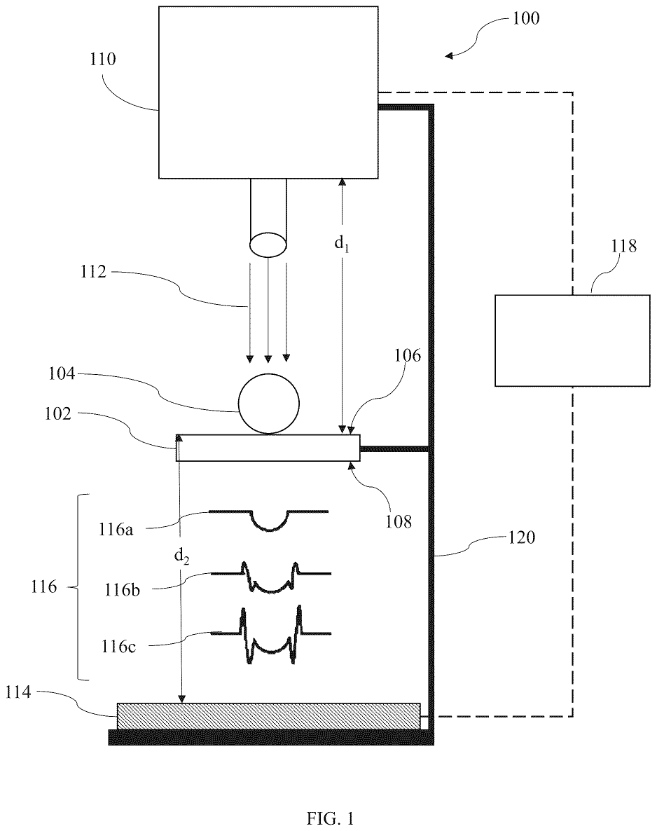

FIG. 1 illustrates a non-limiting embodiment of a phase-contrast enhanced X-ray imaging system 100 in accordance with the present disclosure. The system 100 comprises a substrate 102, an X-ray source 110, an X-ray detector 114, and a control circuit 118. The system 100 can be capable to receive a specimen 104, obtain a phase-contrast enhanced image of the specimen 104, and classify features within the phase-contract enhanced image.

The specimen 104 may be any organic or inorganic compound wherein the microstructure of the specimen 104 can be investigated for medical, industrial, and/or scientific purposes. The specimen 104 may be obtained from a subject, such as, for example, a human or an animal. The specimen 104 can be a tissue sample, such as, for example, a breast tissue sample. The tissue sample can be obtained at a point of care (e.g., a clinic or a hospital). For example, a breast tissue sample can be biopsied from a subject after an initial screening from a physician. The specimen 104 may have been previously obtained and stored for a period of time. The specimen 104 can be obtained from the subject and quickly analyzed thereafter without subjecting the specimen 104 to storage.

The substrate is capable to hold the specimen 104, and the substrate 102 comprises a first side 106 and a second side 108, which may be oppositely disposed from the first side 106. The first side 106 of the substrate 102 can be adjacent to the specimen 104 and/or in contract with the specimen 104. The substrate 102 can be a plate or other surface capable of supporting the specimen 104. The substrate 102 may be cylindrical. The substrate 102 may include walls or an outer perimeter capable of constraining the specimen 104 to the substrate 102. The substrate 102 may be divisible into segments, such that different specimens 104 can be placed in one or more of the segments of the substrate 102. The substrate 102 may be attached to the phase-contrast enhanced X-ray imaging system 100 via a frame 120. The substrate 102 may be moveable or fixed relative to the X-ray source 110 and/or the X-ray detector 114 as the application may require. The substrate 102 may be rotatable, such that different portions of the specimen 104 positioned thereon can be imaged.

The substrate 102 can comprise a material capable of supporting the specimen 104 and enabling substantial transmission of X-ray electromagnetic radiation 112 such that the X-ray detector 114 can receive X-ray radiation 116. The substrate 102 can comprise a radiolucent material. For example, the substrate 102 can comprise a polymeric material, such as, for example, an acrylic polymer, and/or a composite material, such as, for example, carbon fiber.

Referring again to FIG. 1, the X-ray source 110 is positioned a first distance, d₁, from the first side 106 of the substrate 102. The X-ray source 110 is capable of emitting X-ray electromagnetic radiation 112 towards the first side 106 of the substrate 102. The X-ray electromagnetic radiation 112 emitted by the X-ray source 110 may include electromagnetic waves with wavelengths in a range of 10⁻¹² to 10⁻⁸ meters.

The first distance, d₁, can be sufficient for enabling placement of the specimen 104 on the substrate 102 and achieving a desirable focus and field of view of the specimen 104 to produce the phase-contrast enhanced X-ray image thereof. In various non-limiting embodiments, the first distance, d₁, can be at least 5 centimeters, such as, for example, at least 10 centimeters, at least 20 centimeters, at least 30 centimeters, at least 40 centimeters, at least 50 centimeters, at least 100 centimeters, at least 150 centimeters, or at least 200 centimeters. In various non-limiting embodiments, the first distance, d₁, can be no greater than 500 centimeters, such as, for example, no greater than 450 centimeters, no greater than 400 centimeters, no greater than 350 centimeters, no greater than 300 centimeters, no greater than 250 centimeters, or no greater than 200 centimeters. For example, the first distance, d₁, can be in a range of 5 centimeters to 500 centimeters, such as, for example, 10 centimeters to 400 centimeters, 30 centimeters to 300 centimeters, 40 centimeters to 250 centimeters, 50 centimeters to 200 centimeters, or 60 centimeters to 100 centimeters.

The X-ray source 110 may be a vacuum chamber comprising an anode and a cathode. To generate the X-ray electromagnetic radiation 112, high current can be applied to the cathode to increase its temperature, thereby allowing electrons to be released therefrom and accelerated under high voltage toward the anode. When the electrons hit the anode, the electrons can suddenly stop, causing a loss of energy to be released in the form of X-ray photons. This results in a beam of X-ray electromagnetic radiation 112 directed towards the substrate 102.

The X-ray source 110 can be configured to enable a desirable focal spot size for forming a phrase contrast enhanced X-ray image. For example, a small focal spot size can improve the resolution and quality of the resultant X-ray image as compared to X-ray sources with larger focal spot sizes, depending on the geometric magnification. The X-ray source 110 may comprise a focal spot size of no greater than 50 μm, such as, for example, no greater than 30 μm.

Referring again to FIG. 1, the X-ray detector 114 can be positioned a second distance, d₂, from the first side 106 of the substrate 102. The X-ray detector can be opposing the X-ray source 110 in order to receive X-ray electromagnetic radiation transmitted from the X-ray source 110. The X-ray detector 114 can convert the received electromagnetic radiation 116 into an electrical signal that can be used to produce a phase-contrast enhanced X-ray image. The X-ray detector can be an indirect detection detector or a direct detection detector. For example, the X-ray detector can comprise a CMOS sensor, a CCD sensor, a scintillation material, a direct detection material, or a combination thereof.

The positioning of the X-ray source 110 and the X-ray detector 114 can define a field of view for the images that can be produced by the system 100. The specimen 104 can be placed in the field of view on the substrate 102 in order to create a phase-contrast enhanced X-ray image thereof.

The X-ray source 110 can emit X-ray electromagnetic radiation 112 towards the specimen 104 and substrate 102. As the X-ray electromagnetic radiation 112 passes through the specimen 104, the specimen 104 absorbs at least a portion of the X-ray electromagnetic radiation 112 resulting in X-ray electromagnetic radiation 116a. The varying composition of the specimen 104 can result in differing absorption coefficients and/or refraction indices within differing portions of the specimen 104. As a result, different parts of the specimen 104 may absorb and/or refract the X-ray electromagnetic radiation 112 to different degrees.

The X-ray detector 114 is spaced the second distance, d₂, from the first side 106 of the substrate to enable the X-ray electromagnetic radiation 116a that passed through the specimen 104 and/or substrate 102 to form an interference pattern prior to interacting with the X-ray detector 114. For example, as the X-ray electromagnetic radiation 116a moves towards the X-ray detector 114, the X-ray electromagnetic radiation 116a begins to interfere with itself resulting in an interference pattern that is present in X-ray electromagnetic radiation 116b. The interference pattern propagates further as electromagnetic radiation 116b moves towards the X-ray detector 114 and eventually forms X-ray electromagnetic radiation 116c. X-ray electromagnetic radiation 116c includes the interference pattern and an absorption pattern from passing through the specimen 104. The X-ray detector 114 is capable to receive the X-ray electromagnetic radiation 112 and detect the combined interference pattern and absorption pattern.

As the second distance, d₂, increases, the interference pattern 116 can become more pronounced. To obtain a desirable interference pattern 116 to produce a phase-contrast enhanced X-ray image, the distance between the specimen and the X-ray detector (e.g., the second distance d₂) can be selected to enable sufficient propagation of the interference pattern in the X-ray electromagnetic radiation 116, such that the interference pattern 116 develops to a level detectable by the X-ray detector 114. In various non-limiting embodiments, the second distance, d₂, can be at least 25% of the first distance d₁, such as, for example, at least 35%, at least 45%, at least 50%, at least 75%, at least 80%, at least 90%, or at least 100%, all of the first distance, d₁. In various non-limiting embodiments, the second distance, d₂, can be no greater than 2000% of the first distance d₁, such as, for example, no greater than 1500%, no greater than 1000%, no greater than 500%, no greater than 250%, no greater than 200%, no greater than 150%, or no greater than 100%, all of the first distance, d₁. For example, the second distance d₂ can be in a range of 25% to 2000% of the first distance d₁, such as, for example, 35% to 1500%, 45% to 1000%, 50% to 500%, 75% to 250%, or 80% to 200%, all of the first distance, d₁

In various other embodiments, the substrate 102 holding the specimen 104 and/or the X-ray detector 114 may be adjustable, such that the second distance, d₂, can be adjusted to accommodate different sized specimens 104 and/or different X-ray imaging components. For example, the second distance, d₂, may be adjustable to control a field of view of the system 100.

The X-ray detector 114 can have sufficient spatial resolution in order to detect the interference pattern within the X-ray electromagnetic radiation 116. The spatial resolution defines a detector's ability to distinguish between two points in space. Spatial resolution can be based on the spatial frequency at which the modulation transfer function (MTF) is reduced to 10%. X-ray detectors 114 with high MTF are typically better at detecting fine details in small objects as compared to X-ray detectors 114 with low MTF. The X-ray detector 114 may have a spatial resolution of at least 30 lp/mm, such as, for example, at least 40 lp/mm, or at least 50 lp/mm. In various examples, the X-ray detector 11 may have a spatial resolution in a range of 30 lp/mm to 100 lp/mm.

The focal spot size of the X-ray source 110, the second distance, d₂, and the spatial resolution of the X-ray detector 114 can be balanced to create a desirable phrase contrast enhanced X-ray image. For example, if the spatial resolution of the X-ray detector 114 is increased, then the distance d₂ and/or the focal spot size of the X-ray source 110 may be reduced. If the second distance, d₂, is increased, the spatial resolution of the X-ray detector 114 and/or the focal spot size of the X-ray source 110 may be reduced. If the focal spot size of the X-ray source 110 is reduced, the second distance, d₂, and/or the spatial resolution of the X-ray detector 114 may be reduced. The focal spot size of the X-ray source 110, the second distance, d₂, and the spatial resolution of the X-ray detector 114 can be balanced to accommodate the spatial footprint of the system 100, the size of the specimen 104, and/or the availability and cost of each component.

Referring again to FIG. 1, the control circuit 118 can be in signal communication with the X-ray source 110 and the X-ray detector 114. The control circuit 118 can be capable to generate a phase-contrast enhanced X-ray image of the specimen 104 based on the X-ray electromagnetic radiation 116 including the interference pattern. An example of a phase-contrast enhanced X-ray image 300 is shown in FIG. 3. Comparing FIG. 3 to FIG. 2, the phase-contrast enhanced X-ray image 300 illustrates finer details of the microcalcifications 322 compared to the microcalcifications 222 in X-ray image 200. The X-ray image 200 shown in FIG. 2 depicts a first specimen generated using a typical X-ray imaging system in accordance with standard clinical protocols. The focal spot size of the X-ray source, the second distance d₂, and the spatial resolution of the X-ray detector of the X-ray imaging system used to produce the X-ray image 200 shown in FIG. 2 were configured in such a way that an interference pattern could not be detected by the X-ray detector. FIG. 3 shows a phase-contrast enhanced X-ray image 300 of a second, similar specimen produced using a phase-contrast enhanced X-ray imaging system as described by the present disclosure. The phase-contrast enhanced X-ray imaging system used to produce the phase-contrast enhanced X-ray image 300 of FIG. 3 was capable of detecting the interference pattern. By comparing FIG. 3 to FIG. 2, the enhanced resolution of the fine details of the microcalcifications are apparent, highlighting the differences capable of being produced using a phase-contrast enhanced X-ray imaging system according to the present disclosure.

As used herein, the term “control circuit” may refer to, for example, hardwired circuitry, programmable circuitry (e.g., a computer processor comprising one or more individual instruction processing cores, processing unit, processor, microcontroller, microcontroller unit, controller, digital signal processor (DSP), programmable logic device (PLD), programmable logic array (PLA), or FPGA), state machine circuitry, firmware that stores instructions executed by programmable circuitry, and any combination thereof. The control circuit 118 may, be embodied, collectively or individually, as circuitry that forms part of a larger system, for example, an IC, an ASIC, a SoC, a desktop computer, a laptop computer, a tablet computer, a server, a smart phone, etc. Accordingly, as used herein, a “control circuit” can comprise electrical circuitry having at least one discrete electrical circuit, electrical circuitry having at least one IC, electrical circuitry having at least one application-specific IC, electrical circuitry forming a general-purpose computing device configured by a computer program (e.g., a general-purpose computer configured by a computer program that at least partially carries out processes and/or devices described herein or a microprocessor configured by a computer program that at least partially carries out processes and/or devices described herein), electrical circuitry forming a memory device (e.g., forms of RAM), and/or electrical circuitry forming a communications device (e.g., a modem, communications switch, or optical-electrical equipment). The subject matter described herein may be implemented in an analog or digital fashion, or some combination thereof.

The control circuit 118 may be connected to a remote server, such as a cloud computing system. In various examples, the control circuit 118 may be located remotely or, alternatively, the control circuit 118 may be physically integrated in the phase-contrast enhanced X-ray imaging system 100. The control circuit 118 may include a mobile application for use on a mobile computing device, such as a smartphone or a tablet computer, for example. The control circuit 118 may be integrated into a hospital database, such that the resulting phase-contrast enhanced X-ray image and any resulting diagnostics may be shared across a hospital database/platform.

The control circuit 118 may be capable of identifying an anomaly within the phase-contrast enhanced X-ray image. For example, the anomaly can be a microcalcification of the specimen 104. The anomaly can absorb and/or refract X-ray electromagnetic radiation differently than surrounding tissue. Identifying microcalcifications can assist a physician in early detection of breast cancer, as changes and/or certain aspects of microcalcifications may be indicative of cancer development. The control circuit 118 may be able to identify microcalcifications of the specimen 104 based on an analysis of the phase-contrast enhanced X-ray image.

In various non-limiting embodiments, the control circuit 118 may be capable of determining a property of an anomaly in the phase-contrast enhanced X-ray image. For example, the control circuit 118 may determine shape, size, location, and/or composition of the anomaly. In various examples where the anomaly is a macrocalcification, the control circuit 118 can determine, for example, a shape of the microcalcification, a size of the microcalcification, spiculations on the microcalcification, a location of the microcalcification, and/or a composition of the microcalcification. The properties of the anomaly can be used to classify the anomaly into a certain category. The phase-contrast enhanced X-ray image can enable the determination of the properties of the anomaly, which may not have been distinguishable in prior X-ray images that did not include interference pattern detection.

In various non-limiting embodiments, the control circuit 118 may be capable of identifying an amount of spiculations, an angle of spiculations, and/or a location of spiculations on the microcalcifications based on the phase-contrast enhanced X-ray image. Spiculations, which tend to make a microcalcification look “spikey” relative to a non-spiculated microcalcification, can be used to classify the microcalcifications. For example, the microcalcifications may be classified into a first category and a second category that are based on an analysis of the spiculations in the phase-contrast enhanced X-ray image. The first category may be microcalcifications with a low amount of spiculations and the second category can be microcalcifications that have a higher amount of spiculations than the first category. Higher numbers of spiculations can be indicative of a higher likelihood of breast cancer. In various non-limiting embodiments, the classification may be indicative of the type of the microcalcification, such as benign or malignant. In certain non-limiting embodiments, the classification may be used to flag a physician to conduct further testing or analysis. Identifying an amount of spiculations, the angle of spiculations, the location of the spiculations, etc., can enhance cancer screening processes and enable physicians to focus on the analysis of relevant microcalcifications.

Subject demographics can be used when determining the classifications for microcalcifications. For example, the classification of the microcalcification may be further based on data comprising at least one of an age of the subject, a health history of the subject, and a genetic predisposition of the subject.

Although described herein with reference to breast tissue biopsy samples and microcalcifications, the present disclosure further considers using the phase-contrast enhanced X-ray imaging system 100 for imaging any biopsied tissue. The resulting image of the biopsied tissue may be able to identify and/or classify small structures within the tissue to facilitate a medical diagnosis or prescribe additional testing. Further, the present disclosure may be used to produce a phase-contrast enhanced X-ray image of any other specimen, for medical or nonmedical purposes.

The following numbered clauses are directed to various non-limiting embodiments according to the present disclosure:
Clause 1. A phase contrast enhanced x-ray imaging system comprising a substrate capable to hold a specimen and comprising a first side and a second side; an x-ray source positioned a first distance from the first side of the substrate and capable to emit x-ray electromagnetic radiation towards the first side of the substrate; an x-ray detector positioned a second distance from the first side of the substrate and opposing the x-ray source, the second distance enabling x-ray electromagnetic radiation emitted by the x-ray source to form an interference pattern, wherein the x-ray detector is capable to receive and detect the x-ray electromagnetic radiation including the interference pattern; and a control circuit in signal communication with the x-ray source and the x-ray detector, the control circuit capable to generate a phase contrast enhanced x-ray image of the specimen based on the detected x-ray electromagnetic radiation including the interference pattern.
Clause 2. The phase-contrast enhanced X-ray imaging system of Clause 1, wherein the second distance is sufficient such that the interference pattern develops to a level detectable by the x-ray detector.
Clause 3. The phase-contrast enhanced X-ray imaging system of Clause 1 or 2, wherein the second distance is at least 25% of the first distance.
Clause 4. The phase-contrast enhanced X-ray imaging system of any of Clauses 1-3, wherein the second distance is at least 50% of the first distance.
Clause 5. The phase-contrast enhanced X-ray imaging system of any of Clauses 1-4, wherein the second distance is adjustable for controlling a field of view of the specimen.
Clause 6. The phase-contrast enhanced X-ray imaging system of any of Clauses 1-5, wherein the x-ray detector has a spatial resolution of at least 30 lp/mm.
Clause 7. The phase-contrast enhanced X-ray imaging system of any of Clauses 1-6, wherein the x-ray source comprises a focal spot size of no greater than 30 μm.
Clause 8. The phase-contrast enhanced X-ray imaging system of any of Clauses 1-7, wherein the specimen is a tissue sample.
Clause 9. The phase-contrast enhanced X-ray imaging system of any of Clauses 1-8, wherein the tissue sample is a breast biopsy tissue sample.
Clause 10. The phase-contrast enhanced X-ray imaging system of any of Clauses 1-9, wherein the control circuit is capable to identify a microcalcification of the specimen based on the phase contrast enhanced x-ray image.
Clause 11. The phase-contrast enhanced X-ray imaging system of Clause 10, wherein the control circuit is capable to determine a property of the microcalcification of the specimen based on the phase contrast enhanced x-ray image, wherein the property of the microcalcification comprises at least one of a shape of the microcalcification, a size of the microcalcification, a location of the microcalcification, and a composition of the microcalcification.
Clause 12. The phase-contrast enhanced X-ray imaging system of Clause 11, wherein the control circuit is capable to determine a classification of a microcalcification of the specimen based on the property of the microcalcification, wherein the classification comprises a first category of microcalcifications or a second category of microcalcifications.
Clause 13. The phase-contrast enhanced X-ray imaging system of Clause 12, wherein the classification of the microcalcification is further based on data comprising at least one of an age of a subject, a health history of the subject, and a genetic predisposition of the subject.
Clause 14. The phase-contrast enhanced X-ray imaging system of any of Clauses 10-13, wherein the control circuit is capable to identify an amount of spiculations of the microcalcification of the specimen based on the phase contrast enhanced x-ray image.
Clause 15. The phase-contrast enhanced X-ray imaging system of any of Clauses 1-14, wherein a focal spot size of the x-ray source, the second distance, and a spatial resolution of the x-ray detector are configured to enable the control circuit to generate the phase contrast enhanced x-ray image.
Clause 16. A phase contrast enhanced x-ray imaging system comprising a substrate capable to hold a specimen and comprising a first side and a second side; an x-ray source comprising a focal spot size, the x-ray source positioned a first distance from the first side of the substrate and capable to emit x-ray electromagnetic radiation towards the first side of the substrate; an x-ray detector comprising a spatial resolution, the x-ray detector positioned a second distance from a first side of the substrate and opposing the x-ray source, the second distance enabling x-ray electromagnetic radiation emitted by the x-ray source to form an interference pattern, wherein the x-ray detector is capable to receive the x-ray electromagnetic radiation and detect the interference pattern; and a control circuit in signal communication with the x-ray source and the x-ray detector, wherein the focal spot size, the second distance, and the spatial resolution of the x-ray detector are configured to enable the control circuit to generate a phase contrast enhanced x-ray image of the specimen.
Clause 17. The phase-contrast enhanced X-ray imaging system of Clause 16, wherein the phase contrast enhanced x-ray image of the specimen is further based on the detected interference pattern and the received x-ray electromagnetic radiation.
Clause 18. The phase-contrast enhanced X-ray imaging system of Clause 16 or 17, wherein the specimen is a breast biopsy tissue sample.
Clause 19. The phase-contrast enhanced X-ray imaging system of any of Clauses 16-18, wherein the control circuit is capable to identify a microcalcification of the specimen based on the phase contrast enhanced x-ray image.
Clause 20. The phase-contrast enhanced X-ray imaging system of any of Clauses 16-19, wherein the spatial resolution of the x-ray detector is at least 30 lp/mm, the focal spot size of the x-ray source is no greater than 30 μm, and the second distance is at least 25% of the first distance.

Having thus described several aspects and embodiments of the technology of this application, it is to be appreciated that various alterations, modifications, and improvements will readily occur to those of ordinary skill in the art. Such alterations, modifications, and improvements are intended to be within the scope of the technology described in the application. It is, therefore, to be understood that the foregoing embodiments are presented by way of example only and that, within the scope of the appended claims and equivalents thereto, inventive embodiments may be practiced otherwise than as specifically described. In addition, any combinations of two or more features, systems, articles, materials, and/or methods described herein, if such features, systems, articles, materials, and/or methods are not mutually inconsistent, are included within the scope of the present disclosure.

In certain embodiments, a processor may be a physical or virtual processor. In other embodiments, a virtual processor may be spread across one or more portions of one or more physical processors. In certain embodiments, one or more of the embodiments described herein may be embodied in hardware such as a Digital Signal Processor (DSP). In certain embodiments, one or more of the embodiments herein may be executed on a DSP. One or more of the embodiments herein may be programmed into a DSP. In some embodiments, a DSP may have one or more processors and one or more memories. In certain embodiments, a DSP may have one or more computer readable storages. In many embodiments, a DSP may be a custom designed ASIC chip. In other embodiments, one or more of the embodiments stored on a computer readable medium may be loaded into a processor and executed.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or”, as used herein in the specification and in the claims, should be understood to mean “either or both” of the elements so conjoined, i.e., elements that are conjunctively present in some cases and disjunctively present in other cases.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. The transitional phrases “consisting of” and “consisting essentially of” shall be closed or semi-closed transitional phrases, respectively.

Where a range or list of values is provided, each intervening value between the upper and lower limits of that range or list of values is individually contemplated and is encompassed within the disclosure as if each value were specifically enumerated herein. In addition, smaller ranges between and including the upper and lower limits of a given range are contemplated and encompassed within the disclosure. The listing of exemplary values or ranges is not a disclaimer of other values or ranges between and including the upper and lower limits of a given range.

Embodiments disclosed herein may be embodied as a system, method, or computer program product. Accordingly, embodiments may take the form of an entirely hardware embodiment, an entirely software embodiment (including firmware, resident software, micro-code, etc.), or an embodiment combining software and hardware aspects that may all generally be referred to herein as a “circuit”, “module”, or “system”. Furthermore, embodiments may take the form of a computer program product embodied in one or more computer readable medium(s) having computer readable program code embodied thereon.

What is claimed is:

1. A phase-contrast enhanced X-ray imaging system comprising:
a substrate capable to hold a specimen and comprising a first side and a second side;
an X-ray source positioned a first distance from the first side of the substrate and capable to emit X-ray electromagnetic radiation towards the first side of the substrate;
an X-ray detector positioned a second distance from the first side of the substrate and opposing the X-ray source, the second distance enabling X-ray electromagnetic radiation emitted by the X-ray source to form an interference pattern, wherein the X-ray detector is capable to receive and detect the X-ray electromagnetic radiation including the interference pattern; and
a control circuit in signal communication with the X-ray source and the X-ray detector, the control circuit capable to generate a phase-contrast enhanced X-ray image of the specimen based on the detected X-ray electromagnetic radiation including the interference pattern.

2. The phase-contrast enhanced X-ray imaging system of claim 1, wherein the second distance is sufficient such that the interference pattern develops to a level detectable by the X-ray detector.

3. The phase-contrast enhanced X-ray imaging system of claim 2, wherein the second distance is at least 25% of the first distance.

4. The phase-contrast enhanced X-ray imaging system of claim 2, wherein the second distance is at least 50% of the first distance.

5. The phase-contrast enhanced X-ray imaging system of claim 1, wherein the second distance is adjustable for controlling a field of view of the specimen.

6. The phase-contrast enhanced X-ray imaging system of claim 1, wherein the X-ray detector has a spatial resolution of at least 30 lp/mm.

7. The phase-contrast enhanced X-ray imaging system of claim 1, wherein the X-ray source comprises a focal spot size of no greater than 30 μm.

8. The phase-contrast enhanced X-ray imaging system of claim 1, wherein the specimen is a tissue sample.

9. The phase-contrast enhanced X-ray imaging system of claim 8, wherein the tissue sample is a breast biopsy tissue sample.

10. The phase-contrast enhanced X-ray imaging system of claim 1, wherein the control circuit is capable to identify a microcalcification of the specimen based on the phase-contrast enhanced X-ray image.

11. The phase-contrast enhanced X-ray imaging system of claim 10, wherein the control circuit is capable to determine a property of the microcalcification of the specimen based on the phase-contrast enhanced X-ray image, wherein the property of the microcalcification comprises at least one of a shape of the microcalcification, a size of the microcalcification, a location of the microcalcification, and a composition of the microcalcification.

12. The phase-contrast enhanced X-ray imaging system of claim 11, wherein the control circuit is capable to determine a classification of a microcalcification of the specimen based on the property of the microcalcification, wherein the classification comprises a first category of microcalcifications or a second category of microcalcifications.

13. The phase-contrast enhanced X-ray imaging system of claim 12, wherein the classification of the microcalcification is further based on data comprising at least one of an age of a subject, a health history of the subject, and a genetic predisposition of the subject.

14. The phase-contrast enhanced X-ray imaging system of claim 10, wherein the control circuit is capable to identify an amount of spiculations of the microcalcification of the specimen based on the phase-contrast enhanced X-ray image.

15. The phase-contrast enhanced X-ray imaging system of claim 1, wherein a focal spot size of the X-ray source, the second distance, and a spatial resolution of the X-ray detector are configured to enable the control circuit to generate the phase-contrast enhanced X-ray image.

16. A phase-contrast enhanced X-ray imaging system comprising:
a substrate capable to hold a specimen and comprising a first side and a second side;
an X-ray source comprising a focal spot size, the X-ray source positioned a first distance from the first side of the substrate and capable to emit X-ray electromagnetic radiation towards the first side of the substrate;
an X-ray detector comprising a spatial resolution, the X-ray detector positioned a second distance from a first side of the substrate and opposing the X-ray source, the second distance enabling X-ray electromagnetic radiation emitted by the X-ray source to form an interference pattern, wherein the X-ray detector is capable to receive the X-ray electromagnetic radiation and detect the interference pattern; and
a control circuit in signal communication with the X-ray source and the X-ray detector, wherein the focal spot size, the second distance, and the spatial resolution of the X-ray detector are configured to enable the control circuit to generate a phase-contrast enhanced X-ray image of the specimen.

17. The phase-contrast enhanced X-ray imaging system of claim 16, wherein the phase-contrast enhanced X-ray image of the specimen is further based on the detected interference pattern and the received X-ray electromagnetic radiation.

18. The phase-contrast enhanced X-ray imaging system of claim 16, wherein the specimen is a breast biopsy tissue sample.

19. The phase-contrast enhanced X-ray imaging system of claim 16, wherein the control circuit is capable to identify a microcalcification of the specimen based on the phase-contrast enhanced X-ray image.

20. The phase-contrast enhanced X-ray imaging system of claim 16, wherein the spatial resolution of the X-ray detector is at least 30 lp/mm, the focal spot size of the X-ray source is no greater than 30 μm, and the second distance is at least 25% of the first distance.
